(19) United States
(12) Patent Application Publication
(10) Pub. No.: US 2023/0248508 A1
(43) Pub. Date: Aug. 10, 2023

(54) SYSTEMS AND METHODS FOR CMC SUTURE SUSPENSIONPLASTY

(71) Applicant: ExsoMed Corporation, Aliso Viejo, CA (US)

(72) Inventors: Lloyd P. Champagne, Phoenix, AZ (US); Larry Ehmke, Portland, OR (US)

(21) Appl. No.: 18/106,811

(22) Filed: Feb. 7, 2023

Related U.S. Application Data
(60) Provisional application No. 63/267,655, filed on Feb. 7, 2022.

(51) Int. Cl. A61F 2/08; A61B 17/04

(52) U.S. Cl. CPC A61F 2/0811; A61B 17/0401; A61B 2017/0404; A61B 2017/0445; A61B 2017/0464; A61F 2002/0852; A61F 2002/0858; A61F 2002/0882

(57) ABSTRACT

Provided herein are surgical kit systems and a methods for using the same for treating thumb arthritis. The surgical kit system includes a spacer, a strand, a first anchoring mechanism, and a second anchoring mechanism. The spacer is configured to be disposed between the thumb metacarpal and the index metacarpal to prevent impingement between the thumb metacarpal and the index metacarpal. The strand is configured to be anchored to the second and first metacarpals by the first and second anchoring mechanism to suspend the thumb metacarpal, respectively.

### PRIORITY CLAIM

The present application claims priority to and the benefit of U.S. Provisional Pat. Application No. 63/267,655, filed on, Feb. 7, 2022 the entirety of which is incorporated herein by reference.

### BACKGROUND

The carpometacarpal (CMC) joint forms the base of the thumb and is where the metacarpal bone of the thumb, also known as the first metacarpal, attaches to the trapezium bone. Cartilage is found at the base of the bones and acts as a cushion, allowing the bones to glide smoothly against each other. Arthritis of the thumb or CMC arthritis is a common problem that occurs when the cartilage wears away from the ends of the bones of the CMC joint. Without cartilage, the thumb metacarpal and the trapezium bone rub directly against each other, which can cause severe pain, swelling, and decreased strength of the thumb.

Treatment for CMC arthritis can include removal of the trapezium bone in a procedure called a trapeziectomy, which creates space and prevents bone on bone interaction between the thumb metacarpal and the trapezium. However, removal of the trapezium can cause other problems. First, the thumb metacarpal can collapse onto the scaphoid, which is called thumb metacarpal subsidence. To remedy this issue, surgeons can implant natural or synthetic material in place of the trapezium. However, the current methods can cause proximal migration of the thumb metacarpal causing the thumb and the index metacarpal to rub together, which is known as impingement. There is not currently a device or method that treats thumb arthritis and prevents both impingement and subsidence.

### SUMMARY

The present disclosure provides new and innovative surgical kit systems and methods for using the same for treating thumb arthritis and preventing impingement between the thumb and index metacarpals. In some examples, a method of treating and/or preventing thumb arthritis in a patient is provided. The method includes anchoring a first part of a strand to an index metacarpal, disposing a spacer along the strand, and anchoring a second part of the strand to the thumb metacarpal. The spacer is disposed between the thumb metacarpal and the index metacarpal to prevent impingement between the thumb metacarpal and the index metacarpal. The method may further include drilling a hole through the thumb metacarpal and threading a second part of the strand through the hole in the thumb metacarpal before anchoring the strand to the thumb metacarpal.

In some examples, a surgical kit system is provided. The surgical kit system includes a spacer, a strand, a first anchoring mechanism, and a second anchoring mechanism. The spacer is configured to be disposed between an index metacarpal and a thumb metacarpal. The strand includes a first part and a second part. The first part of the strand is configured to be anchored to the index metacarpal by the first anchoring mechanism, and the second part of the strand is configured to be anchored to the thumb metacarpal by the second anchoring mechanism.

Additional features and advantages of the disclosed surgical kit systems and methods are described in, and will be apparent from, the following Detailed Description and the Figures.

### BRIEF DESCRIPTION OF THE FIGURES

FIG. 1 is a diagram of an example surgical kit system according to an example of the present disclosure.

FIG. 2 is a diagram of an example surgical kit system according to another example of the present disclosure.

FIG. 3A is a diagram of an example surgical kit system according to another example of the present disclosure.

FIG. 3B is an expanded view of a first part of a strand of the surgical kit system of FIG. 3A, where a second part of the strand is fed through a hole in the first part. In FIG. 3B, a spacer of the surgical kit system of FIG. 3A is omitted for illustrative purposes.

FIGS. 4A and 4B are diagrams of an example surgical kit system according another example of the present disclosure.

FIGS. 5A-5C are diagrams illustrating an example method according to an example of the present disclosure.

FIG. 6 is a diagram illustrating an example method according to another example of the present disclosure.

FIGS. 7A-7C are diagrams illustrating an example method according to another example of the present disclosure.

FIGS. 8A-8B are diagrams illustrating an example method according to another example of the present disclosure.

FIG. 9 is a diagram of an example surgical kit system according to another example of the present disclosure.

FIGS. 10A-10C are diagrams of an example surgical kit system according to another example of the present disclosure.

### DETAILED DESCRIPTION OF EXAMPLES

The present disclosure is directed to systems and methods for treating thumb arthritis and preventing impingement between the first and second metacarpals. The systems and methods according to the present disclosure may also prevent the thumb metacarpal subsidence.

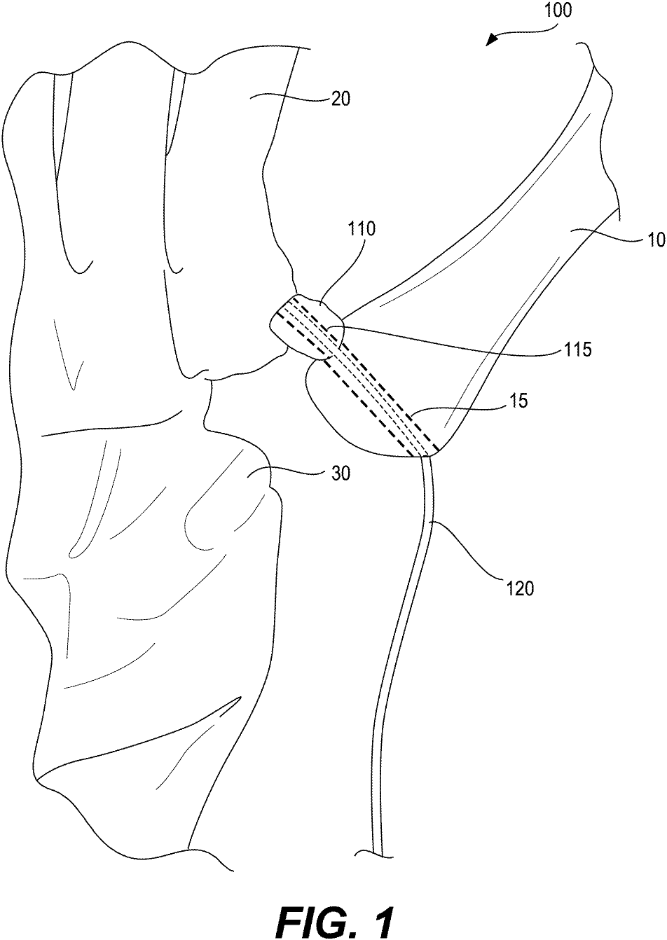

FIG. 1 shows an example surgical kit system 100 according to an example of the present disclosure. The surgical kit system 100 of the present disclosure may include a spacer 110 and a strand 120. In some examples, the spacer 110 may be configured to provide interposition between the first (thumb) metacarpal 10 and the second (index) metacarpal 20.

The strand 120 may be configured to hold the spacer 110 in place and suspend the first metacarpal so as to prevent the first metacarpal 10 from collapsing onto the scaphoid 30 after removal of the trapezium. The strand 120 may include a first part and a second part. In some examples, the first part of the strand 120 may be configured to be anchored to the second metacarpal 20, while the second part of the strand 120 may be configured to be anchored to the first metacarpal 10.

The strand 120 can be any suitable length and can be trimmed during the procedure depending on the desired length and desired tension of the strand 120. The tension of the strand 120 can alter the thumb’s movement, such as the thumb’s abduction or how far the thumb can spread from the midline of the fingers. In some examples, the length of the strand 120 may be the same throughout every surgical kit system. In other examples, the length of the strand 120 can be tailored to the specific patient for whom the surgical kit system will be provided.

Non-limiting examples of the strand 120 may include a string, a thread, a rope, a wire, a tape, a rod, and a suture. The strand 110 can be constructed of or include any suitable biocompatible/medical-grade material. For example, the strand 120 may be made of or include a flexible material, such as (nonabsorbable) suture material. Non-limiting examples of the suture materials may include nylon, prolene, silk, polyester, or any combinations thereof.

In some examples, the strand 120 may be in the form of a tape having a width greater than the thickness. In some examples, the ratio between the width and the thickness of the tape may be in the range of about 2:1 to about 10:1, for example, about 2:1 to about 4:1, about 4:1 to about 6:1, about 6:1 to about 8:1, or about 8:1 to about 10:1. In other examples, the ratio between the width and the thickness of the tape may have any other suitable range. When the strand 120 is in the form of a tape, the strand 120 may be less prone to cut through the bone (e.g., first/second metacarpal), tissues around the bone, and/or the spacer 110 than a strand 120 in the form of a string/wire.

In some examples, the width of the strand 120 may be in the range of about 1 mm to about 10 mm, for example, about 1 mm to about 3 mm, about 3 mm to about 5 mm, about 5 mm to about 7 mm, or about 7 mm to about 10 mm. In some examples, the thickness of the strand 120 may be in the range of about 0.1 mm to about 2.0 mm, for example, about 0.1 mm to about 0.5 mm, about 0.5 mm to about 1.0 mm, about 1.0 mm to about 1.5 mm, about 1.5 mm to about 2.0 mm. In other examples, the strand 120 may have any other suitable width and/or length.

The strand 120 may have a sufficient tensile strength to withstand the force associated with movement of the thumb. In some examples, the tensile strength of the strand 120 may be in the range of about 10 N to about 880 N, for example, about 10 N to about 110 N, about 110 N to about 220 N, about 220 N to about 330 N, about 330 N to about 440 N, about 440 N to about 550 N, about 550 N to about 660 N, about 660 N to about 770 N, or about 770 N to about 880 N. In other examples, the strand 120 may have any other suitable tensile strength.

The spacer 110 may be disposed along the strand 120 so that the spacer 110 lies between the first and second metacarpals. This arrangement may provide interposition and cushioning between the two bones, which may prevent impingement of the first and second metacarpals.

In some examples, the spacer 110 may be cannulated so that the strand 120 threads through the through-hole 115 of the spacer 110. In other examples, the spacer 110 may be tied onto the strand 120. For example, the strand 120 may tie around the outer surface of the spacer 110.

Non-limiting examples of a shape of the spacer 110 may include a cylinder, a prism, a cube, a cuboid, a sphere, an ellipsoid, a cone, a torus, an octahedron, and a disc. In other examples, the spacer 110 may not have a defined shape or may have any other suitable shape. The spacer 110 may be symmetrical or asymmetrical along its length.

In some examples, the spacer 110 may be made of or include a rigid material, such as hard plastics, metals, or any combinations thereof. For example, the spacer 110 may be made of or include a suitable metal (e.g., cobalt, notinol (nickel titanium), stainless steel) and/or a suitable plastic (e.g., polyethylenes, polyetheretherketones (PEEK), polylactic acid copolymers, polyglycolic copolymers).

In other examples, the spacer 110 may be made of or include a soft material like a textile. The textile material can include polyester, polytetrafluoroethylene (PTFE), expanded polytetrafluoroethylene (ePTFE), polyethylene terephthalate (PET) or any other suitable biocompatible-grade material or a combination thereof. In some examples, the spacer 110 may be made of or include a biological material. The biological material can include collagen, (allograft) tendon, muscle, fat, skin, or any other suitable joint interposition material or a combination thereof. In some examples, the spacer 110 may be made out of any other suitable implantable materials, such as polyurethane urea, silicone, and pyrocarbon. The spacer 110 may be made out of combinations of the materials as described herein. In some examples, the spacer 110 may be made of a (woven) suture.

In some examples, the spacer 110 may be one-size fits all for each patient. In other examples, the spacer 110 may be a custom size based on a patient’s anatomy, such as the size of and the space between the first and second metacarpals. A doctor or surgeon can determine the anatomy of the patient by any suitable means, such as by x-ray.

In some examples, the spacer 110 may have a length in the range of about 1 mm to about 10 mm, for example, about 1 mm to about 3 mm, about 3 mm to about 5 mm, about 5 mm to about 7 mm, or about 7 mm to about 10 mm. In other examples, the spacer 110 may have any other suitable length.

In some examples, the surgical kit system 100 can additionally include one or more anchoring mechanisms for anchoring the strand 120 to the bone. The one or more anchoring mechanisms may be configured to fix the strand 120 to the bone, for example, without ripping or damaging the strand. Non-limiting examples of the anchoring mechanism include a fastener, such as a screw (e.g., a tenodesis screw), a (threaded) suture anchor, a button, a flip button, or any other suitable component/device that can fix the strand 120 to bones (e.g., any fastener that can fix the strand 120 to bones without cutting/damaging the strand 120). In some examples, the anchoring mechanism may be made out of or include suitable metals, plastics, or combinations thereof. In other examples, the anchoring mechanism may be made out of or include any suitable biocompatible/medical-grade material, such as suture material. The anchoring mechanism may be made out of combinations of the materials as described herein.

In some examples, the one or more anchoring mechanisms may include a first anchoring mechanism configured to anchor the first part of the strand 120 to the second metacarpal and a second anchoring mechanism configured to anchor the second part of the strand 120 to the first metacarpal. The first and second anchoring mechanisms may be the same or may be different.

In some examples, the first anchoring mechanism may anchor the first part of the strand 120 on the radial side of the second metacarpal 20 or the ulnar side of the second metacarpal 20. In other examples, the first anchoring mechanism may anchor the first part of the strand 120 on any other suitable portion of the second metacarpal 20.

In some examples, the first metacarpal 10 may include a hole 15 configured to receive the strand. In some examples, the second part of the strand 120 may be threaded through the hole 15 in the first metacarpal 10 as shown in FIG. 1. In some examples, instead of threading the second part of the strand 120 through a hole, the second part of the strand 120 may be suspended under the first metacarpal 10, acting as a sling under the first metacarpal 10, and anchored to the radial side of the first metacarpal 10.

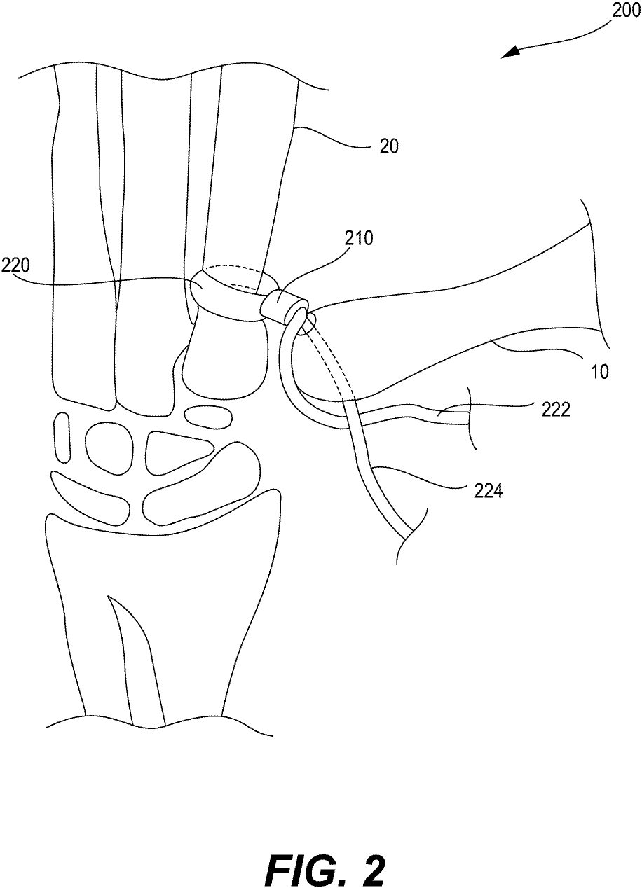

FIG. 2 shows an example surgical kit system 200 according to another example of the present disclosure. The surgical kit system 200 may include a spacer 210 and a strand 220. As shown in FIG. 2, in some examples, the strand 220 may be anchored to the second metacarpal 20 by wrapping the strand 220 around the second metacarpal 20. Anchoring the strand 120 in this manner may result in two parts (a third part 222 and a fourth part 224) of the strand projecting from the radial side of the second metacarpal 20. In some examples, both of the third and fourth parts 222, 224 of the strand 120 may be threaded through the spacer 110 as shown in FIG. 2A. In other examples, both of the third and fourth parts 222, 224 of the strand 220 may be used to tie the spacer 210 onto the strand 220.

In some examples, the third part 222 of the strand 220 may be threaded through the hole 15 of the first metacarpal 10, and the fourth part 224 of the strand 120 may not be threaded through the hole 15. In other examples, both of the third and fourth parts 222, 224 of the strand 220 may or may not be threaded through the hole 15 of the first metacarpal 10.

In some examples, the third and fourth parts 222, 224 of the strand 220 may be anchored to the first metacarpal 10, for example, by knotting the third and fourth parts 222 around the first metacarpal 10 or by using one or more anchoring mechanisms.

Other configurations/features/characteristics of the surgical kit system 200 of FIG. 2 may be similar to and/or the same as the ones described above with respect to the surgical kit system 100 of FIG. 1 (e.g., material, shape, size, function, etc.), and, thus, duplicate description may be omitted.

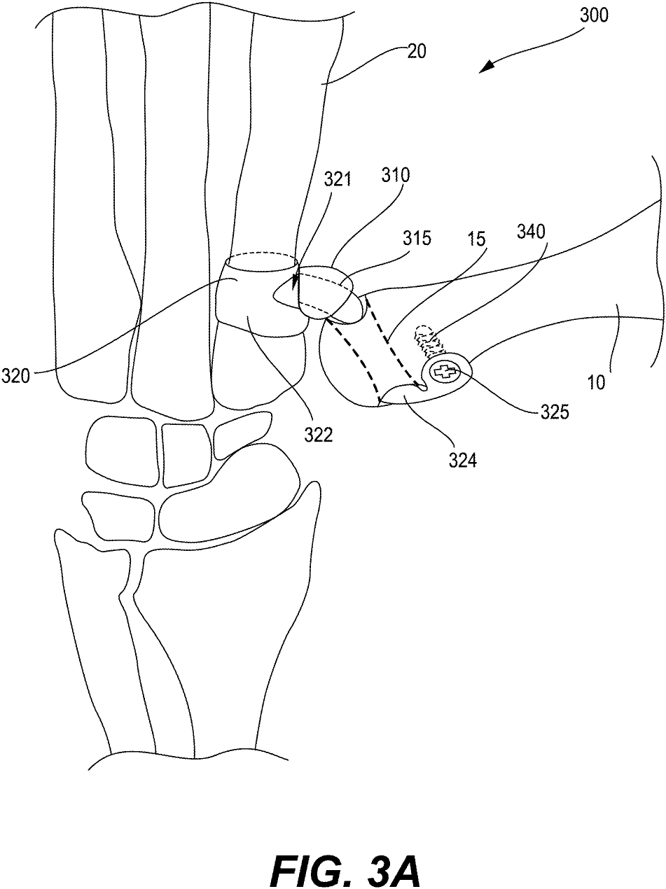

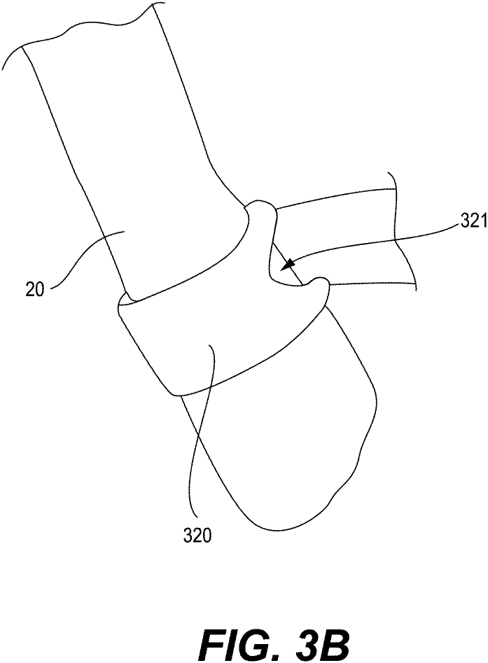

FIG. 3 shows an example surgical kit system 300 according to another example of the present disclosure. The surgical kit system 300 may include a spacer 310 and a strand 320. As shown in FIGS. 3A and 3B, in some examples, the strand 320 may be anchored to the second metacarpal 20 by wrapping the strand 320 around the second metacarpal 20. In some examples, the strand 320 may include a hole 322 in a first part 322. A second part 324 of the strand 320 may be fed through the hole 321 as shown in FIG. 3, which may allow the strand 320 to self-tighten.

In some examples, the second part 324 of the strand 320 may be threaded through the spacer 310 and/or the hole 15 of the first metacarpal 10. In some examples, the strand 120 may include an anchoring mechanism receiving hole 325 in the second part 324, and the second part 324 may be anchored to the first metacarpal 10 with an anchoring mechanism 340.

Other configurations/features/characteristics of the surgical kit system 300 of FIG. 3 may be similar to and/or the same as the ones described above with respect to the surgical kit system 100 of FIG. 1 (e.g., material, shape, size, function, etc.), and, thus, duplicate description may be omitted.

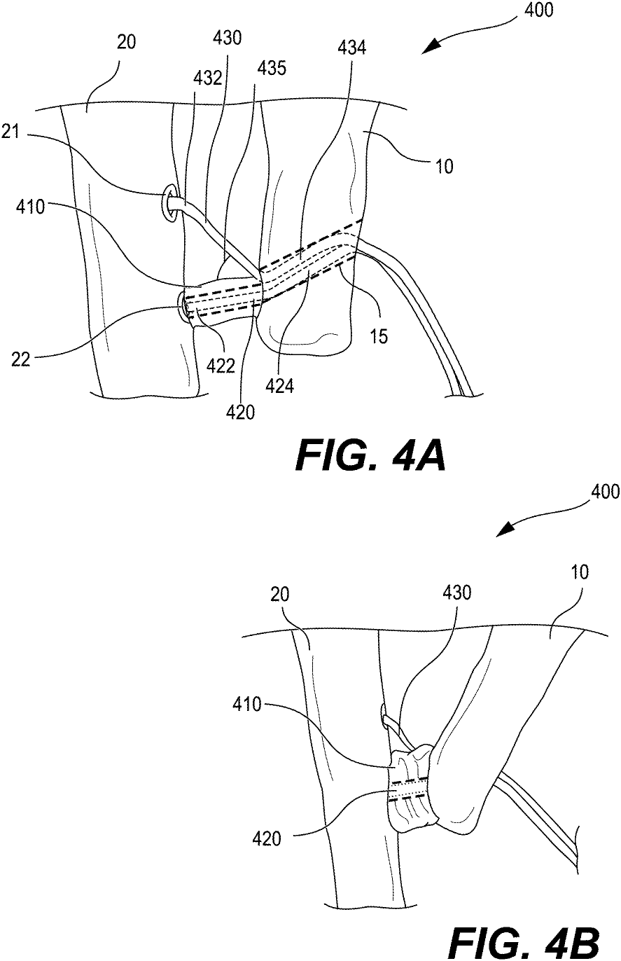

FIGS. 4A and 4B show an example surgical kit system 400 according to another example of the present disclosure. The surgical kit system 400 may include a spacer 410 and a first strand 420. The surgical kit system 400 in FIG. 4A is similar to the surgical kit system 400 in FIG. 4B. One difference between the surgical kit system 400 in FIG. 4A and the surgical kit system 400 in FIG. 4B is that the spacer 410 of FIG. 4A may have a rigid body (e.g., hard plastic or metal) while the spacer 410 of FIG. 4B may have a soft body (e.g., textile).

In some examples, the surgical kit system 400 may include a second strand 430. The second strand 430 may include a first part 432 and a second part 434. The first part 432 of the second strand 430 may be configured to be anchored to the second metacarpal 20 and the second part 434 may be configured to be anchored to the first metacarpal 10.

In some examples, the second part 434 of the second strand 430 and/or the second part 424 of the first strand 420 may be threaded through the hole 15 of the first metacarpal 10. In some examples, the first part 432 of the second strand 430 may be configured to be anchored to the second metacarpal 10 at a first location 21 above a second location 22 at which the first part 422 of the first strand 420 is anchored so that the second metacarpal 10, the spacer 410, and the second strand 430 may form a triangle shape (or substantially in the shape of a triangle). The second strand 430 may provide additional support for the suspension of the first metacarpal 10.

In some examples, an angle 435 formed between the spacer 410 and the second strand 430 may be in the range of about 10° to about 80°, for example, about 10° to about 30°, about 30° to about 50°, about 50° to about 70°, about 70° to about 80°. In other examples, the angle 435 may have any other suitable angle.

Other configurations/features/characteristics of the surgical kit system 400 of FIGS. 4A and 4B may be similar to and/or the same as the ones described above with respect to the surgical kit systems 100, 200, and 300 of FIGS. 1, 2, 3A, and 3B (e.g., material, shape, size, function, etc.), and, thus, duplicate description may be omitted.

In some examples, the components of a surgical kit system according to the present disclosure (e.g., the surgical systems described herein) may be packaged together in a sterile packaging. In some examples, the surgical kit system may be standardized to suit any patient. In other examples, the surgical kit system may be custom made dependent on the patient’s anatomy.

In some examples, a method of treating thumb arthritis using a surgical kit system disclosed herein according to the present disclosure is provided. The method may treat thumb arthritis or CMC arthritis while also avoiding both of impingement of the first and second metacarpals and subsidence of the thumb into the scaphoid. The steps presented herein may be performed in any suitable order and combination, and may be modified by or combined with any of the other procedures and features disclosed elsewhere herein.

In some examples, the method for treating thumb arthritis may include removing the trapezium. Removal of the trapezium may stop the bone on bone interaction between the first metacarpal and the trapezium in patients with degraded cartilage in the CMC joint. The methods disclosed herein can also be performed with the trapezium bone still in place.

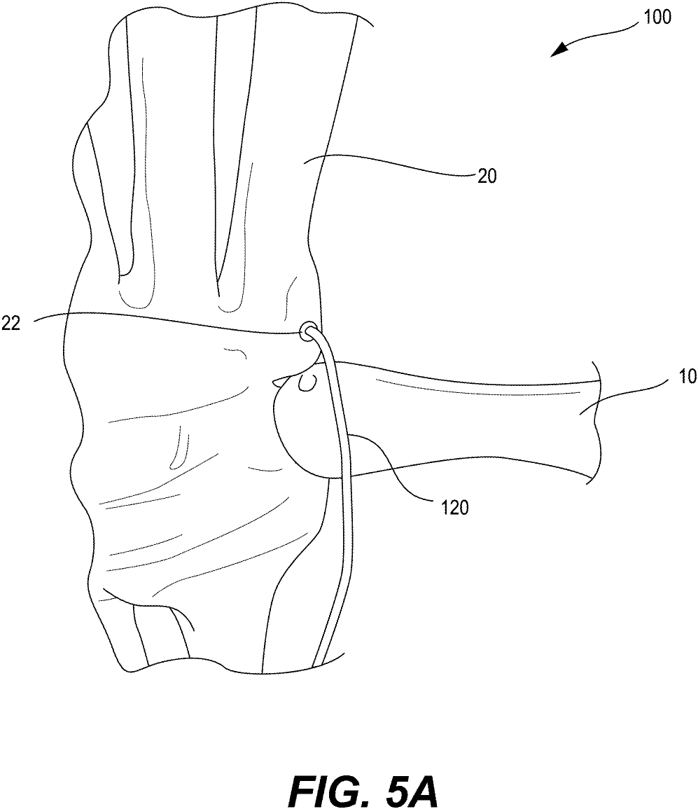

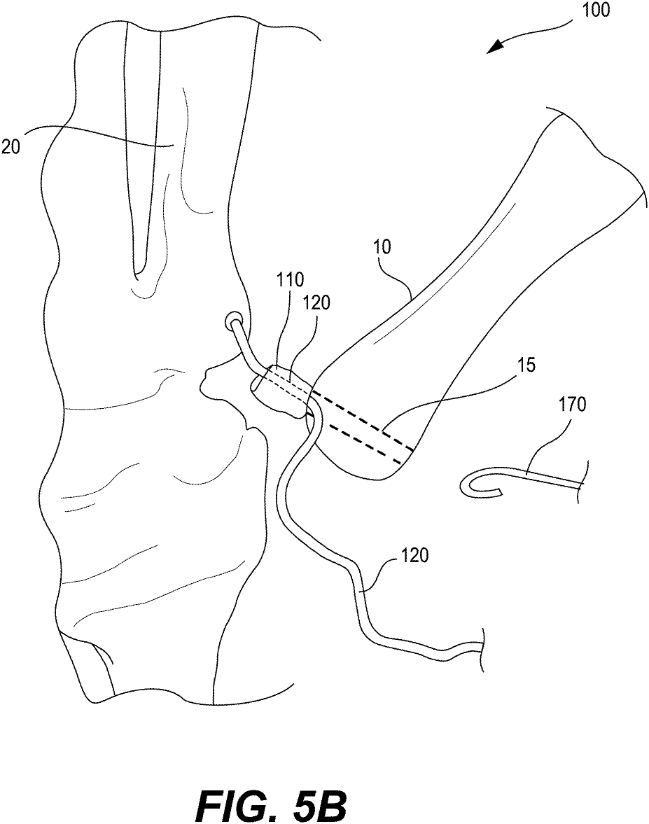

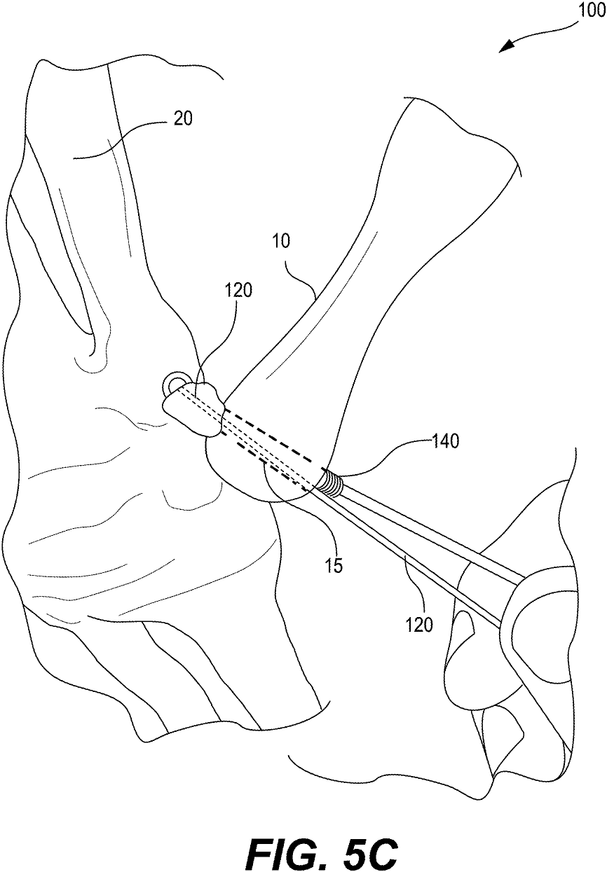

Referring to FIGS. 5A to 5C, the method may include anchoring a first part of a strand 120 to the second metacarpal 20. The strand 120 may be anchored to the second metacarpal by any suitable anchoring mechanism. In some examples, the surgeon may anchor the strand 120 to the radial side of the second metacarpal 20, which may allow the procedure to be done with only one incision instead of two. In some examples, the surgeon may anchor the strand 120 to the ulnar side of the second metacarpal 20. The strand 120 can be anchored in the bottom half of the second metacarpal 20, the base of the second metacarpal 20, or for example, location 22. A surgeon can anchor the strand 120 to the second metacarpal 20 using an anchoring mechanism as discussed herein. For example, a first part of the strand 120 can be anchored to the second metacarpal using a fastener, such as a (tenodesis) screw, a button, or a suture anchor.

In some examples, the strand may be anchored to the second metacarpal 20 by wrapping the strand around the second metacarpal 20 as illustrated in FIG. 2. In some examples, the strand 120 may be anchored to the second metacarpal 20 by wrapping a first part of the strand around the second metacarpal 20, and threading a second part of the strand through a hole on a first part of the strand as shown in FIG. 3.

In some examples, the method may include drilling a hole in the second metacarpal 20, for example, prior to anchoring the strand 120 to the second metacarpal 20. A depth of the hole drilled in the second metacarpal 20 may be substantially the same as a length of the anchoring mechanism (e.g., length of a screw or a suture anchor). The first part of the strand 120 can enter the hole on the second metacarpal 20 prior to anchoring.

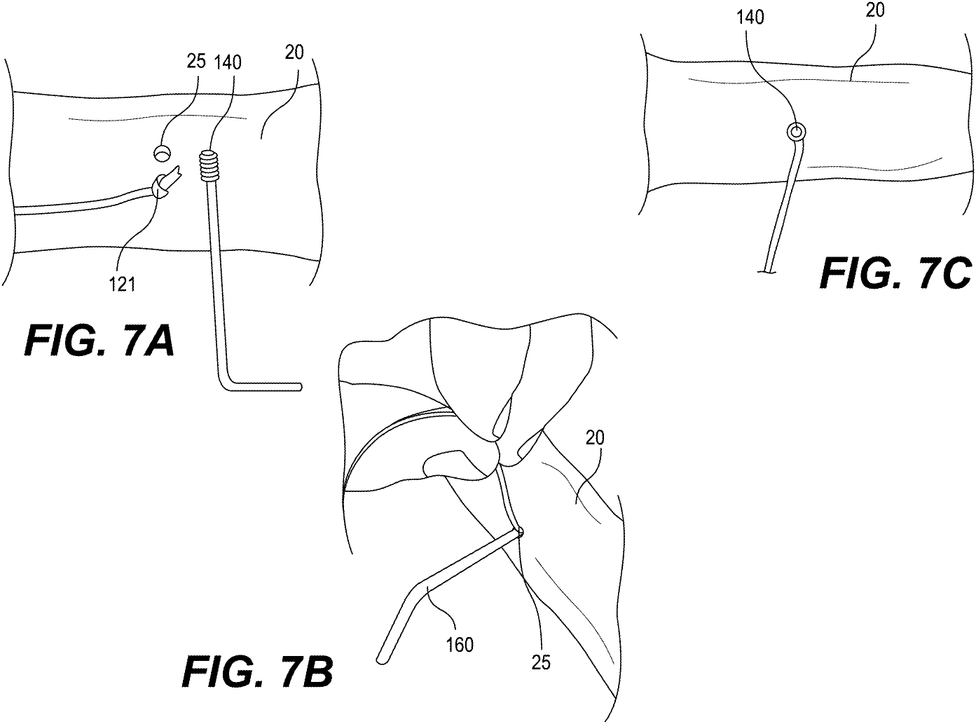

In some examples, a knot may be tied into the first part of the strand 120 before threading the strand 120 into the hole of the second metacarpal 20. For example, referring to FIGS. 7A to 7C, a knot 121 may be formed in the first part of the strand 120, and inserted into the hole 25 on the second metacarpal 20, for example, using a pusher/driver 160. Then, an anchoring mechanism 140 (e.g., a tenodesis screw) may be inserted into the hole 25. In some examples, a first portion of the first part of the strand 120 may be disposed between the anchoring mechanism 140 and the hole 25 of the second metacarpal 20. In some examples, the diameter of the hole 25 may be approximately the same as the diameter of the knot 121 tied in the strand 120.

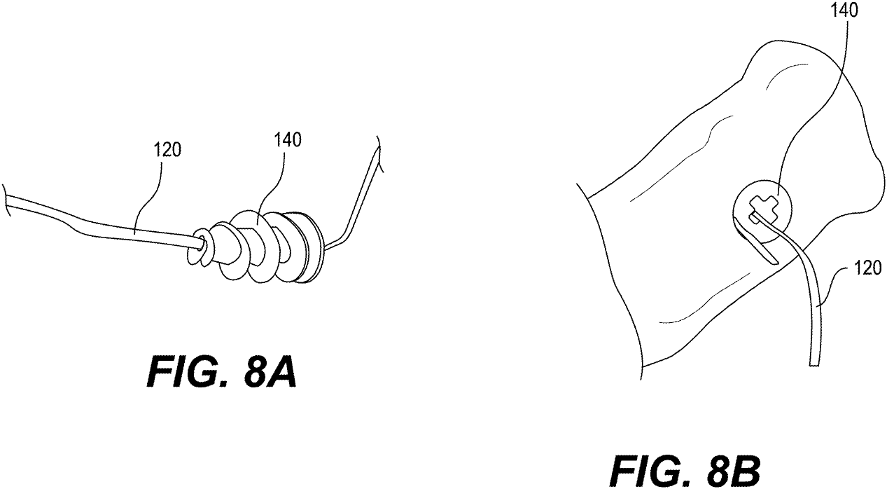

Referring to FIGS. 8A and 8B, in some examples, the anchoring mechanism 140 may be cannulated. In this case, a second portion of the first part of the strand 120 may be disposed within the hole of the anchoring mechanism 140 after the installation of the anchoring mechanism 140 on the bone.

In some examples, the method may include drilling a hole through the diameter of the second metacarpal 20. In some examples, the anchoring mechanism may be disposed through the second metacarpal 20. The hole may be drilled through the second metacarpal 20 volar to dorsal, dorsal to volar, ulnar to radial, or radial to ulnar. In some examples, the strand 120 may be affixed to the anchoring mechanism, and the anchoring mechanism and the strand 120 may be pushed through the hole in the second metacarpal 20. The anchoring mechanism can anchor the strand 120 to the second metacarpal 20 through the hole. In some examples, the strand 120 may be affixed to the anchoring mechanism and just the strand 120 may be pushed through the hole in the second metacarpal 20. In some examples, the method may include disposing the spacer 110 along the strand 120. The spacer 110 may be disposed along the strand 120 before anchoring the first part of the strand 120 to the second metacarpal 20 or after anchoring the first part of the strand 120 to the second metacarpal 20.

The spacer 110 may be disposed along the strand 120 by any suitable method. For example, referring back to FIG. 1, in some examples, the spacer 110 may be cannulated so that the second part of the strand 120 may thread through the hole 115 of the spacer 110. In other examples, the spacer 110 may be tied onto the strand 120.

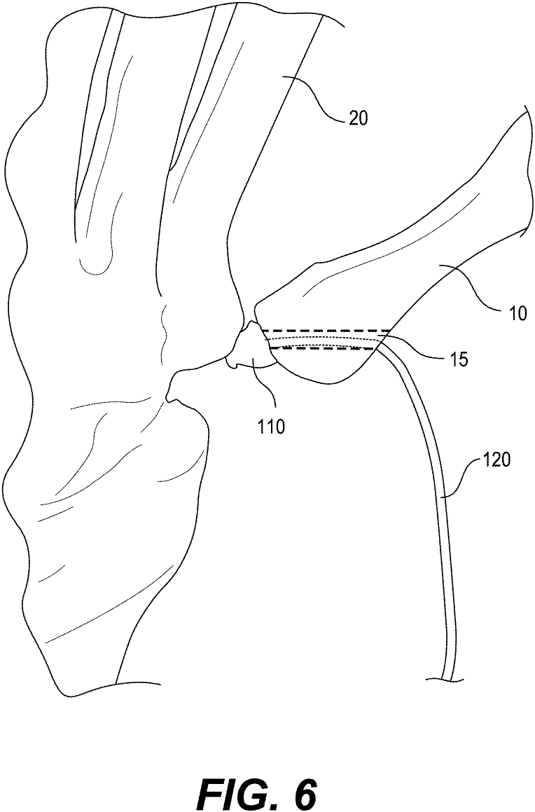

In some examples, the first metacarpal 10 is suspended. The first metacarpal may be suspended by any suitable method. For example, referring back to FIGS. 5B and 5C, in some examples, the method may include drilling a hole through the diameter of the first metacarpal 10 to create a channel 15 through the bone. In some examples, the hole 15 can be drilled through the first metacarpal 10 ulnar to radial or radial to ulnar as shown in FIGS. 5B and 5C. In other examples, the hole can be drilled from the bottom of the first metacarpal 10 out the radial side, as shown in FIG. 6.

After the hole 15 is drilled, the method may include threading the second part of the strand 120 through the channel 15 in the first metacarpal 10. The second part of the strand 120 may be threaded through the channel 15 using any known technique. For example, the strand 120 may be threaded through the channel by inserting a tool 170 with a hook through the channel 15, hooking the strand 120, and pulling the strand 120 back through the channel 15. In some examples, a hole 15 is not drilled into the first metacarpal 10, and the strand 120 is suspended under the first metacarpal 10 acting as a sling under the first metacarpal 10.

As shown in FIG. 5C, once the strand 120 is threaded through the channel 15 or suspended under the first metacarpal, the surgeon may pull the strand 120 tight to suspend the first metacarpal 10. The tensioning of the strand 120 may be important to ensure proper abduction of the thumb and mobility after the procedure. In some examples, the method may further include adjusting the tension of the strand 120 to ensure proper thumb suspension. The adjusting the tension of the strand 120 may occur before anchoring the strand 120 to the first metacarpal 10 or during the anchoring of the strand 120.

After the second part of the strand 120 is threaded through the channel 15 or suspended under the first metacarpal 10, the second part of the strand 120 may be anchored to the first metacarpal 10 using any suitable anchoring method described herein. For example, the second part of the strand 120 may be anchored to the radial side of the first metacarpal 10 using an anchoring mechanism 140. In other examples, the strand 120 may be anchored by tying the strand 120 around the first metacarpal 10. In some examples, the second part of the strand 120 may be anchored in the channel 15 through which the strand 120 is threaded, as shown in FIG. 5C.

In some examples, a second hole is drilled into the first metacarpal 10 above or below the channel 15. A depth of the second hole may be substantially the same as the length of the anchoring mechanism (e.g., length of a screw or suture anchor). In some examples, the strand may be anchored in the second hole. In some examples, a knot (similar to the knot 121 in the first part) may be tied in the second part of the strand 120, the knot may be inserted into the second hole, and an anchoring mechanism may be inserted into the second hole of the first metacarpal 10.

Referring back to FIGS. 4A and 4B, in some examples, the method may include anchoring a first part 432 of a second strand 430 to the second metacarpal 20 at a first location 21 above a second location 22 at which the first part 422 of the first strand 420 is anchored, threading a second part 434 of the second strand 430 through the hole 15 in the first metacarpal 10, and anchoring the second part 434 of the second strand 430 to the first metacarpal 10. The second metacarpal 20, the spacer 410, and the second strand 430 may form a triangle shape as shown in FIGS. 4A and 4B.

Referring back to FIG. 2, in some examples, the method of treating thumb arthritis may include wrapping the strand 220 around the second metacarpal 20 so that a third part 222 and a fourth part 224 of the strand 220 are projected from the radial side of the second metacarpal 20, disposing the spacer 210 along both parts 222, 224 of the strand 120, and anchoring both parts 222, 224 of the strand 210 to the first metacarpal 10.

The spacer 210 can be disposed along the strand by threading both parts 222, 224 of the strand 220 through a cannula bore of the spacer 210. In other examples, one part of the strand 220 may be disposed along one side of the spacer 210 and the other part of the strand 220 may be disposed along the other side of the spacer 210, and both parts of the strand 220 may be tied together to hold the spacer 210 in place.

Once the spacer 210 is in place, in some examples, one part 222 of the strand 220 can be threaded through the channel 15 in the first metacarpal 10, while the other part 224 of the strand 120 may be disposed under the base of the first metacarpal 10. In other examples, both parts of the strand 220 may be threaded through the channel in the first metacarpal 10. The two parts of the strand 220 can be anchored to the first metacarpal 10 by tying the two parts of the strand together or by using an anchoring mechanism as discussed herein.

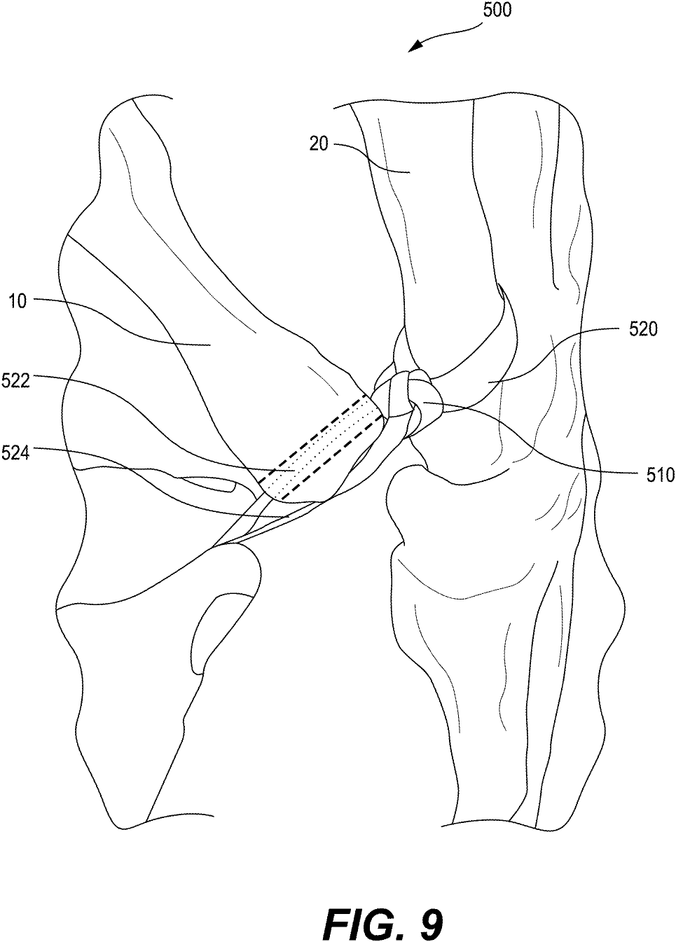

FIG. 9 shows an example surgical kit system 500 according to another example of the present disclosure. The surgical kit system 500 may include a spacer 510 and a strand 520. As shown in FIG. 9, in some examples, the strand 520 may be anchored to the second metacarpal 20 by wrapping the strand 520 around the second metacarpal 20. Anchoring the strand 520 in this manner may result in two parts (a third part 522 and a fourth part 524) of the strand 520 projecting from the radial side of the second metacarpal 20.

In some examples, the third part 522 and the fourth part 524 of the strand 520 may be tied to form a knot, which may function as a spacer 510. In some examples, the knot may be an overhand knot, a surgeons knot or a square knot. In other examples, the knot may be any other suitable knot.

In some examples, the third part 522 of the strand 520 may be threaded through the hole 15 of the first metacarpal 10, and the fourth part 524 of the strand 520 may not be threaded through the hole 15. In other examples, both of the third and fourth parts 522, 524 of the strand 220 may or may not be threaded through the hole 15 of the first metacarpal 10.

In some examples, the third and fourth parts 522, 524 of the strand 520 may be anchored to the first metacarpal 10, for example, by knotting the third and fourth parts 522 around the first metacarpal 10 or by using one or more anchoring mechanisms. In other examples, the third and fourth parts 522, 524 of the strand 520 may be tied to form another knot to fix the strand 520 to the first metacarpal 10.

Other configurations/features/characteristics of the surgical kit system 500 of FIG. 9 may be similar to and/or same as the ones described above with respect to the surgical kit systems 100, 200, 300, and/or 400 of FIGS. 1, 2, 3A, 3B, 4A, and 4B (e.g., material, shape, size, function, etc.), and, thus, duplicate description may be omitted.

In some examples, a surgeon can tie one or more knots (similar to the knot 510) in the strand to be disposed between the first and second metacarpals in addition to a spacer (e.g., the spacer 110, 210, 310, and/or 410 described with respect to FIGS. 1, 2, 3A, 3B, 4A, and 4B). In this case, the one or more knots in the strand may create extra space between the first and second metacarpals 10, 20 in addition to the length of the spacer. In this way, the one or more knots can be used to adjust the distance between the first and second metacarpals 10, 20, for example, when using a one-size fits all spacer. For example, if the spacer is too small for a specific patient, the one or more knots can be tied before or after disposing the spacer on the strand.

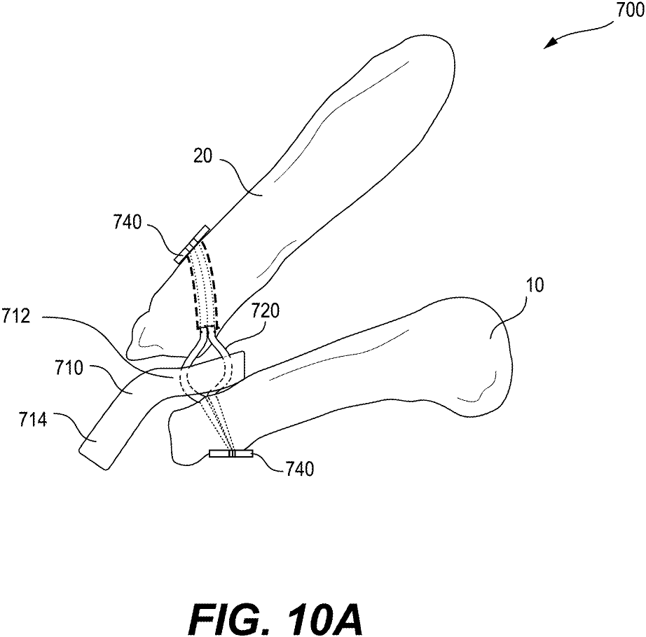

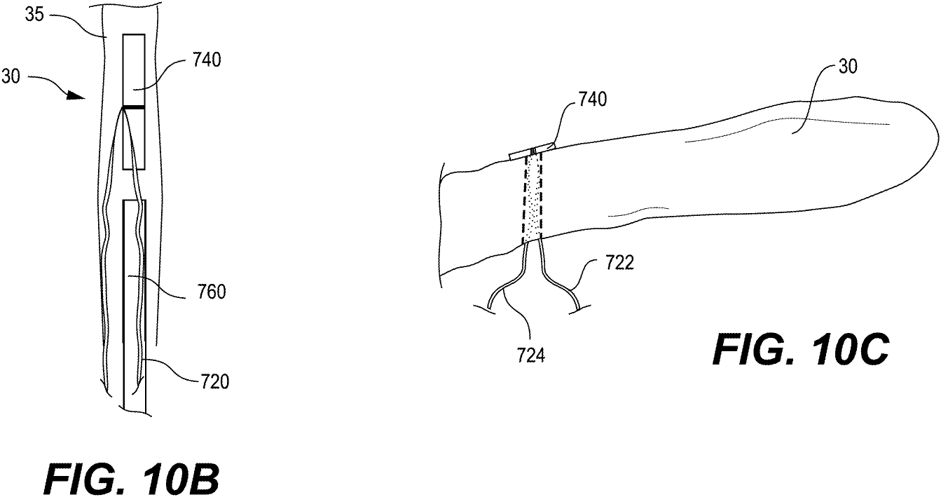

FIG. 10A shows an example surgical kit system 700 according to another example of the present disclosure. The surgical kit system 700 may include a spacer 710 and a strand 720. The spacer 710 may include a first portion 712 configured to be disposed between the first metacarpal 10 and the second metacarpal 20, and a second portion 714 configured to be disposed between the first metacarpal 10 and the scaphoid 30. In some examples, the spacer 710 may be curved or bent.

Referring to FIGS. 10B and 10C, in some examples, the strand 720 may be anchored to the first metacarpal 10 and/or the second metacarpal 20 through an anchoring mechanism 740. For example, the anchoring mechanism 740 may be a flip button and the strand 720 may be tied to the flip button. The flip button 740 may be configured to be pushed through a hole 35 in a bone 30 (the first metacarpal 10 and/or the second metacarpal 20) in a first direction, and upon exiting the hole 35, the flip button 740 may be flipped perpendicular to the first direction (and lying parallel to the bone).

In some examples, the strand 720 may have a third part 722 and a fourth part 724 projecting from the flip button 740. In some examples, the spacer 710 can be disposed along the strand 720 by threading both parts 722, 724 of the strand 720 through a cannula bore of the spacer 710. In other examples, one part 722 of the strand 720 may be disposed along one side of the spacer 710 and the other part 724 of the strand 720 may be disposed along the other side of the spacer 710, and both parts of the strand 720 may be tied together to hold the spacer 710 in place.

In some examples, the anchoring mechanism 740 may be a button and the strand 720 may be tied to the button. The button may be disposed on one side of the bone (the first metacarpal 10 and/or the second metacarpal 20) and the strand 720 may be threaded through a channel in the bone so that the strand 720 is projecting out of the other side of bone opposite the side with the button.

In some examples, the spacer 710 can be disposed along the strand 720 by threading both parts 722, 724 of the strand 720 through a cannula bore of the spacer 710. In other examples, one part 722 of the strand 720 may be disposed along one side of the spacer 710 and the other part 724 of the strand 720 may be disposed along the other side of the spacer 710, and both parts of the strand 720 may be tied together to hold the spacer 710 in place.

In some examples, once the spacer is disposed along the strand 720, the strand 720 is anchored to the first and/or second metacarpal 10, 20. The strand 720 may be anchored to the first and/or second metacarpal 10, 20 by using a button. The strand 720 may be threaded through a hole in the first and/or second metacarpal 10, 20 and the strand may be tied to a button that acts to anchor the strand 720 to the bone.

Other configurations/features/characteristics of the surgical kit system 700 of FIG. 10A may be similar to and/or the same as the ones described above with respect to the surgical kit systems 100, 200, 300, and/or 400 of FIGS. 1, 2, 3A, 3B, 4A, and 4B (e.g., material, shape, size, function, etc.), and, thus, duplicate description may be omitted.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

### Embodiments

Various aspects of the subject matter described herein are set out in the following numbered embodiments:

Embodiment 1. A method of treating thumb arthritis, the method comprising: anchoring a first part of a strand to an index metacarpal; disposing a spacer along the strand; and anchoring a second part of the strand to a thumb metacarpal, wherein the spacer is disposed between the thumb metacarpal and the index metacarpal to prevent impingement between the thumb metacarpal and the index metacarpal.

Embodiment 2. The method of embodiment 1, further comprising: drilling a hole through the thumb metacarpal; and threading a second part of the strand through the hole in the thumb metacarpal.

Embodiment 3. The method of any one of embodiments 1-2, wherein the anchoring of the first part of the strand and/or the second part of the strand is done with one or more anchoring mechanisms.

Embodiment 4. The method of any one of embodiments 1-3, further comprising tying at least one knot in the strand, wherein the at least one knot is disposed between the thumb metacarpal and the index metacarpal.

Embodiment 5. The method of any embodiments 1-4, further comprising: tying a knot in the first part of the strand; inserting the knot into a hole in the index metacarpal; and inserting an anchoring mechanism into the hole in the index metacarpal.

Embodiment 6. The method of any one of embodiments 1-5, further comprising: anchoring a first part of a second strand to the index metacarpal at a first location above a second location at which the first part of the strand is anchored; anchoring the second part of the second strand to the thumb metacarpal, wherein the index metacarpal, the spacer, and the second strand form a triangle shape.

Embodiment 7. The method of any one of embodiments 1-6, wherein the spacer comprises at least one of a plastic material, a metal material, a woven suture, a textile, and a biological material.

Embodiment 8. The method of any one of embodiments 1-7, wherein the strand comprises a tape having a width greater than a thickness thereof.

Embodiment 9. The method of any one of embodiments 1-8, wherein the spacer comprises a knot.

Embodiment 10. The method of any one of embodiments 1-9, wherein the knot comprises at least one of an overhand knot, a surgeons knot, and a square knot.

Embodiment 11. A surgical kit system for treating thumb arthritis, the surgical kit comprising: a spacer configured to be disposed between a thumb metacarpal and an index metacarpal; a strand having a first part configured to be anchored to the index metacarpal and a second part configured to be anchored to the thumb metacarpal; a first anchoring mechanism configured to anchor the first part of the strand to the index metacarpal; and a second anchoring mechanism configured to anchor the second part of the strand to the thumb metacarpal.

Embodiment 12. The surgical kit of embodiment 11, wherein the first anchoring mechanism and/or the second anchoring mechanism comprises a suture anchor.

Embodiment 13. The surgical kit system of embodiment 12, wherein the first anchoring mechanism and/or the second anchoring mechanism is a flip button.

Embodiment 14. The surgical kit system of any one of embodiments 11-13, wherein the strand comprises suture material.

Embodiment 15. The surgical kit system of any one of embodiments 11-14, wherein the strand comprises a tape having a width greater than a thickness thereof.

Embodiment 16. The surgical kit system of any one of embodiments 11-15, wherein the spacer comprises at least one of a plastic material, a metal material, a woven suture, a textile, and a biological material.

Embodiment 17. The surgical kit system of any one of embodiments 11-16, The surgical kit of claim 11, wherein the spacer is cannulated.

Embodiment 18. The surgical kit system of any one of embodiments 11-17, further comprising a second strand having a first part configured to be anchored to the index metacarpal and a second part configured to be anchored to the thumb metacarpal, wherein the first part of the second strand is configured to be anchored to the index metacarpal at a first location above a second location at which the first part of the strand is anchored so that the index metacarpal, the spacer, and the second strand form a triangle shape.

Embodiment 19. The surgical kit system of any one of embodiments 11-18, wherein the spacer comprises a knot.

Embodiment 20. The surgical kit system of embodiment 11-19, wherein a shape of the spacer is one of a cylinder, a cube, a cuboid, a sphere, an ellipsoid, a cone, a torus, and an octahedron.

As used herein, “about,” “approximately” and “substantially” are understood to refer to numbers in a range of numerals, for example the range of -10% to +10% of the referenced number, preferably -5% to +5% of the referenced number, more preferably -1% to +1% of the referenced number, most preferably -0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of treating thumb arthritis, the method comprising:
anchoring a first part of a strand to an index metacarpal;
disposing a spacer along the strand; and
anchoring a second part of the strand to a thumb metacarpal,
wherein the spacer is disposed between the thumb metacarpal and the index metacarpal to prevent impingement between the thumb metacarpal and the index metacarpal.

2. The method of claim 1, further comprising:
drilling a hole through the thumb metacarpal; and
threading a second part of the strand through the hole in the thumb metacarpal.

3. The method of claim 1, wherein the anchoring of the first part of the strand and/or the second part of the strand is done with one or more anchoring mechanisms.

4. The method of claim 1, further comprising tying at least one knot in the strand, wherein the at least one knot is disposed between the thumb metacarpal and the index metacarpal.

5. The method of claim 1, further comprising:
tying a knot in the first part of the strand;
inserting the knot into a hole in the index metacarpal; and
inserting an anchoring mechanism into the hole in the index metacarpal.

6. The method of claim 1, further comprising:
anchoring a first part of a second strand to the index metacarpal at a first location above a second location at which the first part of the strand is anchored;
anchoring the second part of the second strand to the thumb metacarpal,
wherein the index metacarpal, the spacer, and the second strand form a triangle shape.

7. The method of claim 1, wherein the spacer comprises at least one of a plastic material, a metal material, a woven suture, a textile, and a biological material.

8. The method of claim 1, wherein the strand comprises a tape having a width greater than a thickness thereof.

9. The method of claim 1, wherein the spacer comprises a knot.

10. The method of claim 9, wherein the knot comprises at least one of an overhand knot, a square knot, and a surgeons knot.

11. A surgical kit system for treating thumb arthritis, the surgical kit system comprising:
a spacer configured to be disposed between a thumb metacarpal and an index metacarpal;
a strand having a first part configured to be anchored to the index metacarpal and a second part configured to be anchored to the thumb metacarpal;
a first anchoring mechanism configured to anchor the first part of the strand to the index metacarpal; and
a second anchoring mechanism configured to anchor the second part of the strand to the thumb metacarpal.

12. The surgical kit system of claim 11, wherein the first anchoring mechanism and/or the second anchoring mechanism comprises a suture anchor.

13. The surgical kit system of claim 11, wherein the first anchoring mechanism and/or the second anchoring mechanism is a flip button.

14. The surgical kit system of claim 11, wherein the strand comprises suture material.

15. The surgical kit system of claim 11, wherein the strand comprises a tape having a width greater than a thickness thereof.

16. The surgical kit system of claim 11, wherein the spacer comprises at least one of a plastic material, a metal material, a woven suture, a textile, and a biological material.

17. The surgical kit system of claim 11, wherein the spacer is cannulated.

18. The surgical kit system of claim 11, further comprising a second strand having a first part configured to be anchored to the index metacarpal and a second part configured to be anchored to the thumb metacarpal, wherein the first part of the second strand is configured to be anchored to the index metacarpal at a first location above a second location at which the first part of the strand is anchored so that the index metacarpal, the spacer, and the second strand form a triangle shape.

19. The surgical kit system of claim 11, wherein the spacer comprises a knot.

20. The surgical kit system of claim 11, wherein a shape of the spacer is one of a cylinder, a cube, a cuboid, a sphere, an ellipsoid, a cone, a torus, and an octahedron.
